(19) United States
(12) Patent Application Publication
(10) Pub. No.: US 2024/0347178 A1
(43) Pub. Date: Oct. 17, 2024

(54) SYSTEMS AND METHODS FOR COORDINATING CARE VIA AN INTEGRATED SOCIAL DETERMINANTS OF HEALTH (SDOH) PLATFORM

(71) Applicant: MEDecision, Inc, Dallas, TX (US)

(72) Inventor: Kenneth YOUNG, Blue Bell, PA (US)

(21) Appl. No.: 18/133,623

(22) Filed: Apr. 12, 2023

(51) Int. Cl. G16H 40/20; G16H 50/30

(52) U.S. Cl. CPC G16H 40/20; G16H 50/30

(57) ABSTRACT

Methods, non-transitory computer readable media, and care coordination systems are disclosed that output to a care manager device a member view interface comprising member data for a member, service data associated with historical SDoH-related activities for the member, and a selectable SDoH referral element. Referral data, submitted via an SDoH referral form output to the care manager device in response to a selection of the SDoH referral element, is obtained. A program dashboard with selectable program indications for programs, identified by analyzing a stored directory of service provider data based on the referral data, is output to the care manager device. A service request is sent to a service provider server using stored connection data in the service provider data for a service provider associated with one of the programs, in response to a selection of one of the program indications, to initiate a service on behalf of the member.

### TECHNICAL FIELD

This technology generally relates to patient care coordination and management systems and, more particularly, to systems and methods for coordinating care using social determinants of health (SDoH) data in conjunction with patient and care plan data to perform analytics functions that consider demographic, clinical, and SDoH data together to more accurately determine next-best action candidates and assist patients, care managers and service providers in orchestrating support services.

### BACKGROUND

The majority of healthcare is consumed by a relatively small percentage of the population. Within this small subset of the population, at-risk individuals with multiple chronic illnesses lack proper housing, reliable transportation, healthy food sources, employment, access to proper healthcare, and safe living conditions. While these social determinants of health (SDoH) may be non-medical, they make addressing medical issues more complex, and create barriers to effectively achieve optimal health outcomes, while driving up costs for health plans and health care managers.

Efficient and effective care coordination remains elusive for health plans and health care managers due to manual, tedious processes and referral tasks, poor quality data, and/or inadequate workflow technologies, for example. Current care coordination technologies do not elucidate imminent needs for members or patients and do not provide efficient connections to third-party service providers or community-based organizations to carry out referral programs relating to SDoH within reasonable turnaround times. Thus, current technologies (e.g., legacy electronic medical record (EMR), registration, financial, and/or claims systems) fail to address SDoH, resulting in gaps in care, higher utilization rates, higher health care costs, and reduced health outcomes for patients.

### SUMMARY

In one example, a method for coordinating care via an integrated social determinants of health (SDoH) platform is disclosed that includes generating and outputting to a care manager device a member view interface comprising member data for a member associated with a care manager user of the care manager device, service data associated with historical SDoH-related activities for the member, and a selectable SDoH referral element. Referral data submitted via an SDoH referral form generated and outputted to the care manager device in response to a received selection of the SDoH referral element is obtained.

A program dashboard with selectable program indications for one or more programs, identified by analyzing a stored directory of service provider data based on the referral data, is then generated and outputted to the care manager device. A service request is sent to a service provider server using stored connection data in the service provider data for a service provider associated with one of the programs, in response to a received selection of one of the program indications corresponding to the one of the programs, to initiate a service on behalf of the member.

In another example, a care coordination system is disclosed that includes memory including programmed instructions stored thereon and one or more processors configured to execute the stored programmed instructions to generate and output to a care manager device via one or more communication networks for display on a display device a member view interface comprising member data for a member associated with a care manager user of the care manager device, service data associated with historical SDoH-related activities for the member, and a selectable SDoH referral element. Referral data submitted via an SDoH referral form generated and outputted to the care manager device via the communication networks for display on the display device in response to a received selection of the SDoH referral element is obtained.

A program dashboard with selectable program indications for one or more programs, identified by filtering a stored directory of service provider data based on the referral data, is generated and output to the care manager device via the communication networks for display on the display device. A service request is sent via the communication networks to a service provider server associated with one of the programs using an application programming interface (API), in response to a received selection of one of the program indications associated with the one of the programs, to initiate a service on behalf of the member.

In yet another example, a non-transitory computer readable media is disclosed that has instructions for coordinating care via an integrated SDoH platform stored thereon that includes executable code that, when executed by one or more processors, causes the one or more processors to generate and output to a care manager device a member view interface comprising member data for a member associated with a care manager user of the care manager device, service data associated with historical SDoH-related activities for the member, and a selectable SDoH referral element. Referral data submitted via an SDoH referral form generated and outputted to the care manager device in response to a received selection of the SDoH referral element is obtained. The referral data comprises one or more of a service type, a service category, or a service time.

A program dashboard with selectable program indications for one or more programs, identified by filtering a stored directory of service provider data based on the referral data, is generated and outputted to the care manager device. A service request is then sent to a service provider server using stored connection data in the service provider data for a service provider associated with one of the programs, in response to a received selection of one of the program indications, to initiate a service on behalf of the member.

This technology provides a number of advantages including methods, non-transitory computer readable media, and care coordination systems that advantageously integrate social referrals into care management technology workflows. In particular, this technology ingests and synthesizes multiple types and formats of data to create a clinical and financial picture of individual members and member populations. Care managers can leverage real-time, analytics-driven insights based on the ingested data to identify and close high-priority gaps and focus on members' most immediate needs with knowledge of the users' unique constraints and capabilities.

Specifically, the disclosed technology optimizes the delivery of personalized, predictive, and prescriptive social referrals for clinicians, physicians, consumers and their caregivers. This technology also leverages clinical integration, care coordination, and patient population management to provide faster, more efficient referrals to healthcare and related services. Moreover, the disclosed technology uses real-time documentation of patient care and secure, bidirectional communication with automatic referral management and authorization tools to enhance care management, maximize efficiency, and close gaps in care caused by SDoH, thereby removing barriers to good health, lowering utilization rates, and reducing health care costs.

### BRIEF DESCRIPTION OF THE DRAWINGS

FIG. 1 is a block diagram of an exemplary network environment with a care coordination system, care manager devices, and service provider devices;

FIG. 2 is a block diagram of an exemplary care coordination system;

FIG. 3 is a flowchart of an exemplary method for coordinating care via an integrated social determinants of health (SDoH) platform;

FIG. 4 is a screenshot of an exemplary member detail interface;

FIG. 5 is a screenshot of an exemplary care manager portal;

FIG. 6 is a screenshot of an exemplary member view interface;

FIG. 7 is a flowchart of an exemplary method for obtaining and processing an SDoH referral to engage a service provider;

FIG. 8 is a screenshot of an exemplary SDoH referral form;

FIG. 9 is a screenshot of an exemplary program dashboard;

FIG. 10 is a screenshot of the exemplary program dashboard of FIG. 9 with a program detail overlay;

FIG. 11 is a screenshot of an exemplary service request form;

FIG. 12 is a screenshot of an exemplary referral dashboard;

FIG. 13 is a screenshot of an exemplary referral update form; and

FIG. 14 is a screenshot of an operational dashboard interface.

### DETAILED DESCRIPTION

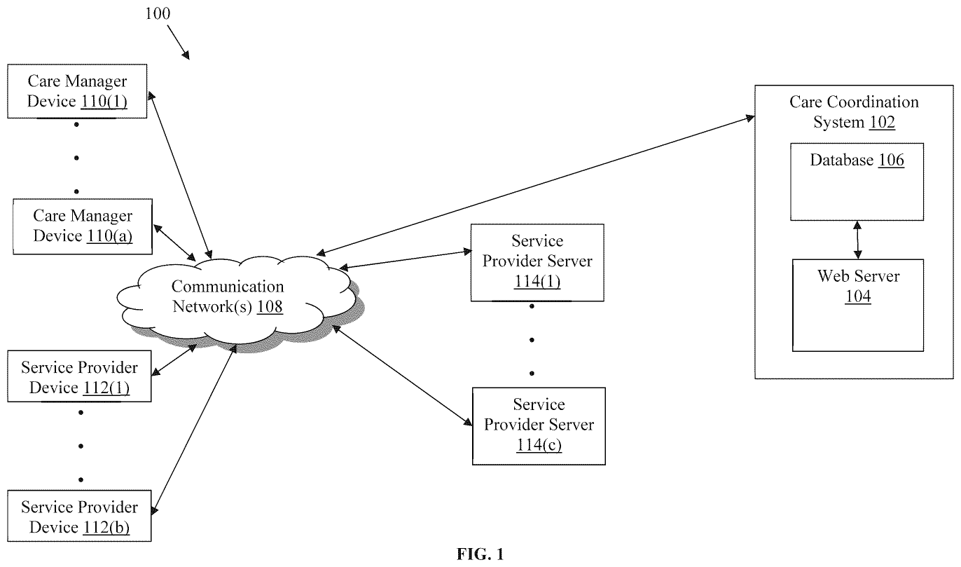

Referring to FIG. 1, an exemplary network environment is illustrated that includes a care coordination system 102, with a web server 104 coupled to a backend database 106, which is coupled via communication network(s) 108 to care manager devices 110(1)-110(a), service provider devices 112(1)-112(b), and service provider servers 114(1)-114(c). The network environment may include other network devices such as one or more routers or switches, for example, which are known in the art and thus will not be described herein. This technology has a number of advantages including methods, non-transitory computer readable media, and care coordination systems that provide an integrated social determinants of health (SDoH) platform with improved graphical interfaces and service provider integrations to facilitate real-time SDoH referrals and service updates to close the SDoH gaps in care caused by SDoH.

In this particular example, the care manager devices 110(1)-110(a), service provider devices 112(1)-112(b), service provider servers 114(1)-114(c), web server 104, and database 106 are disclosed in FIG. 1 as dedicated hardware devices. However, one or more of the care manager devices 110(1)-110(a), service provider devices 112(1)-112(b), service provider servers 114(1)-114(c), web server 104, or database 106 can also be implemented in software within one or more other devices in the network environment. As one example, the database 106, as well as any of its components or applications, can be implemented as software executing on the web server 104, and many other permutations and types of implementations and network topologies can also be used in other examples.

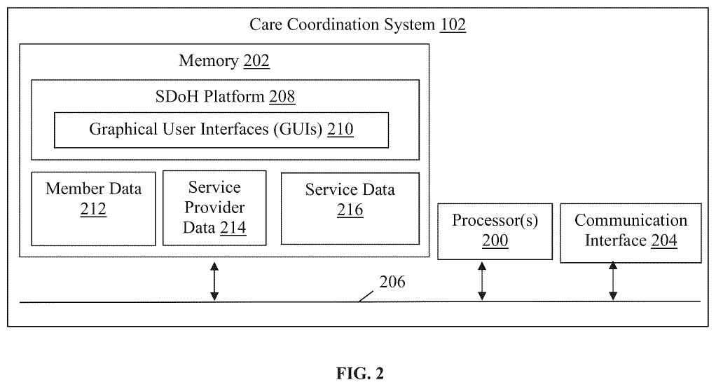

Referring to FIGS. 1-2, the care coordination system 102 may perform any number of functions, including providing graphical user interfaces (GUIs) to the care manager devices 110(1)-110(a) and service provider devices 112(1)-112(b) to facilitate presentations of aggregated and processed data regarding members, initiation of services by service providers on behalf of members, and perform other functions as described and illustrated in detail below. The care coordination system 102 (e.g., the web server 104 of the care coordination system 102) in this example includes processor(s) 200, memory 202, and a communication interface 204, which are coupled together by a bus 206, although the care coordination system 102 can include other types or numbers of elements in other configurations.

The processor(s) 200 of the care coordination system 102 may execute programmed instructions stored in the memory 202 of the care coordination system 102 for any number of the functions described and illustrated herein. The processor(s) 200 may include one or more general purpose processors with one or more processing cores, one or more central processing units, and/or one or more graphics processing units, for example, although other types of processor(s) can also be used.

The memory 202 stores these programmed instructions for one or more aspects of the present technology as described and illustrated herein, although some or all of the programmed instructions could be stored elsewhere. A variety of different types of memory storage devices, such as random-access memory (RAM), read only memory (ROM), hard disk, solid state drives, flash memory, or other computer readable medium which is read from and written to by a magnetic, optical, or other reading and writing system that is coupled to the processor(s) 200, can be used for the memory 202.

Accordingly, the memory 202 can store applications that can include computer executable instructions that, when executed by the processor(s) 200, cause the care coordination system 102 to perform actions, such as to transmit, receive, or otherwise process network messages and requests, for example, and to perform other actions described and illustrated below. The application(s) can be implemented as components of other applications, operating system extensions, and/or plugins, for example.

Further, the application(s) may be operative in a cloud-based computing environment with access provided via a software-as-a-service (SaaS) model. The application(s) can be executed within or as virtual machine(s) or virtual server(s) that may be managed in a cloud-based computing environment. Also, the application(s), and even the care coordination system 102 itself, may be located in virtual server(s) running in a cloud-based computing environment rather than being tied to specific physical network computing devices. Also, the application(s) may be running in virtual machines (VMs) executing on the care coordination system 102 and managed or supervised by a hypervisor.

In this particular example, the memory 202 includes an SDoH platform 208 with GUIs 210, member data 212, service provider data 214, and service data 216. In some examples, the member data 212, service provider data 214, and service data 216 can be maintained in the database 106 of the care coordination system 102, which can be a relational database (e.g., a Structured Query Language (SQL) database), although other types of databases can also be used in other examples.

The SDoH platform 208 is configured to generate and provide the GUIs 210 to facilitate interfacing with the care manager devices 110(1)-110(a), service provider devices 112(1)-112(b), and service provider servers 114(1)-114(c). For example, the SDoH platform 208 obtains the member data 212 from the care manager devices 110(1)-110(a) and facilitates initiation of SDoH services on behalf of members or patients associated with particular care managers. The member data 212 can include demographic data, a generated unique identifier, an indication of SDoH needs or SDoH base data, one or more SDoH risk scores, clinical data, and/or an indication of an associated care manager (e.g., a unique identifier of the care manager), although other member data 212 can also be used in other examples.

The SDoH platform 208 can initiate the SDoH service based on referral and other data received from the care manager devices 110(1)-110(n) by interfacing with the service provider servers 114(1)-114(c) (e.g., using provided application programming interfaces (APIs) or other endpoints or connection information maintained in the service provider data 214). The service provider servers 114(1)-114(c) can be hosted by SDoH service providers (e.g., community-based organizations (CBO) that provide SDoH services (e.g., transportation, food, pharmacy, or behavioral health services). Additionally, the service provider data 214 can include a directory of programs offered by each of a plurality of identified service providers.

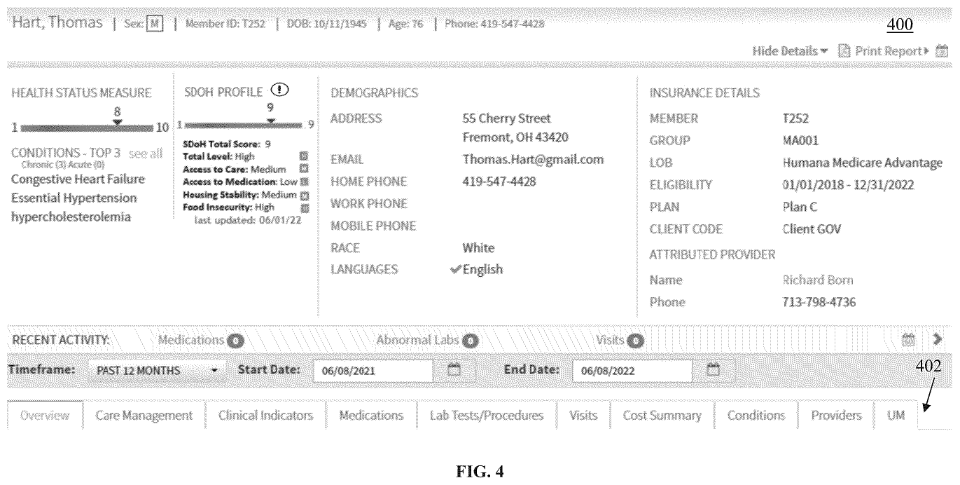

Once initiated, the SDoH platform 208 stores the referral data and other information regarding the initiated SDoH service in the service data 216 and facilitates communications and status updates regarding the SDoH services to inform the care manager, for example, regarding the status of the service. The status updates can also be maintained in the service data 216. Accordingly, one or more of the GUIs 210 can be provided to the care manager devices 110(1)-110(a) (e.g., for initiating an SDoH referral) and/or the service provider devices 112(1)-112(b) (e.g., for accepting and updating the status of the SDoH referral), and exemplary GUIs 210 will be described and illustrated in more detail below with reference to FIGS. 4-6 and 8-14.

The communication interface 204 of the care coordination system 102 operatively couples and communicates between the care coordination system 102, care manager devices 110(1)-110(a), service provider devices 112(1)-112(b), and service provider servers 114(1)-114(c), which are coupled together at least in part by the communication network(s) 108 in this particular example, although other types or numbers of communication networks or systems with other types or numbers of connections or configurations to other devices or elements can also be used. The communication network(s) 108 can include wide area network(s) (WAN(s)) and/or local area network(s) (LAN(s)), for example, and can use TCP/IP over Ethernet and industry-standard protocols, although other types or numbers of protocols or communication networks can be used. The communication network(s) 108 can employ any suitable interface mechanisms and network communication technologies including, for example, Ethernet-based Packet Data Networks (PDNs).

The care coordination system 102 in some examples can include a plurality of devices each having one or more processors (each processor with one or more processing cores) that implement one or more steps of this technology. In these examples, one or more of the devices can have a dedicated communication interface or memory. Alternatively, one or more of the devices can utilize the memory 202, communication interface 204, or other hardware or software components of one or more other devices included in the care coordination system 102. Additionally, one or more of the devices that together comprise the care coordination system 102 in other examples can be standalone devices or integrated with one or more other devices or apparatuses.

Each of the service provider servers 114(1)-114(c) can include processor(s), memory, and a communication interface, which are coupled together by a bus or other communication link (not illustrated), although other numbers or types of components could also be used. The service provider servers 114(1)-114(c) can be associated with or hosted by SDoH service providers, such as Uber, Instacart™, Talkspace™, Care.com™, Walmart™, Door Dash™, Amazon Pharmacy™, and/or any other provider of transportation, food, pharmacy, or behavioral health services.

Accordingly, the service provider servers 114(1)-114(c) can provide API endpoints, for example, that intake referral requests with particular portions of the service data 216 (e.g., time or location of the SDoH service). With the received data, the service provider servers 114(1)-114(c) are configured to facilitate engagement of a particular service provider (e.g., via communication with the service provider devices 112(1)-112(b)) and provide data regarding the engagement back to the care coordination system 102 in response to the referral request, as explained in more detail below.

Each of the care manager devices 110(1)-110(a) and service provider devices 112(1)-112(b) of the network environment 100 in this example includes any type of computing device that can exchange network data, such as mobile, desktop, laptop, or tablet computing devices, virtual machines (including cloud-based computers), or the like. Each of the care manager devices 110(1)-110(a) and service provider devices 112(1)-112(b) in this example includes processor(s), memory, and a communication interface, which are coupled together by a bus or other communication link (not illustrated), although other numbers or types of components could also be used.

Each of the care manager devices 110(1)-110(a) and service provider devices 112(1)-112(b) may run interface applications, such as web browsers or standalone applications, which may provide an interface to communicate with the care coordination system 102 via the communication network(s) 108. Each of the care manager devices 110(1)-110(a) and service provider devices 112(1)-112(b) may further include a display device, such as a display screen or touchscreen, or an input device, such as a keyboard or mouse, for example (not shown). One or more of the care manager devices 110(1)-110(a) and service provider devices 112(1)-112(b) may be associated with an individual user (e.g., a care manager or a service provider), for example.

Accordingly, the care manager devices 110(1)-110(a) can be used by care managers to interface with the care coordination system 102 to manage care for associated members. Additionally, the service provider devices 112(1)-112(b) can be used by service providers to accept referral requests sent via the service provider servers 114(1)-114(c) and update SDoH service statuses, for example, as will be described and illustrated in more detail below.

Although the exemplary network environment 100 with the care manager devices 110(1)-110(a), service provider devices 112(1)-112(b), service provider servers 114(1)-114(c), care coordination system 102, and communication network(s) 108 are described and illustrated herein, other types or numbers of systems, devices, components, or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the components depicted in the network environment, such as the care manager devices 110(1)-110(a), service provider devices 112(1)-112(b), service provider servers 114(1)-114(c), or care coordination system 102, for example, may be configured to operate as virtual instances on the same physical machine. In other words, one or more of the care manager devices 110(1)-110(a), service provider devices 112(1)-112(b), service provider servers 114(1)-114(c), or care coordination system 102 may operate on the same physical device rather than as separate devices communicating through the communication network(s) 108. Additionally, there may be more or fewer care manager devices, service provider devices, service provider servers, or care coordination systems than illustrated in FIG. 1.

The examples of this technology may also be embodied as one or more non-transitory computer readable media having instructions stored thereon, such as in the memory 202 of the care coordination system 102, for one or more aspects of the present technology, as described and illustrated by way of the examples herein. The instructions in some examples include executable code that, when executed by one or more processors, such as the processor(s) 200 of the care coordination system 102, cause the processors to carry out steps necessary to implement the methods of the examples of this technology that will now be described and illustrated herein.

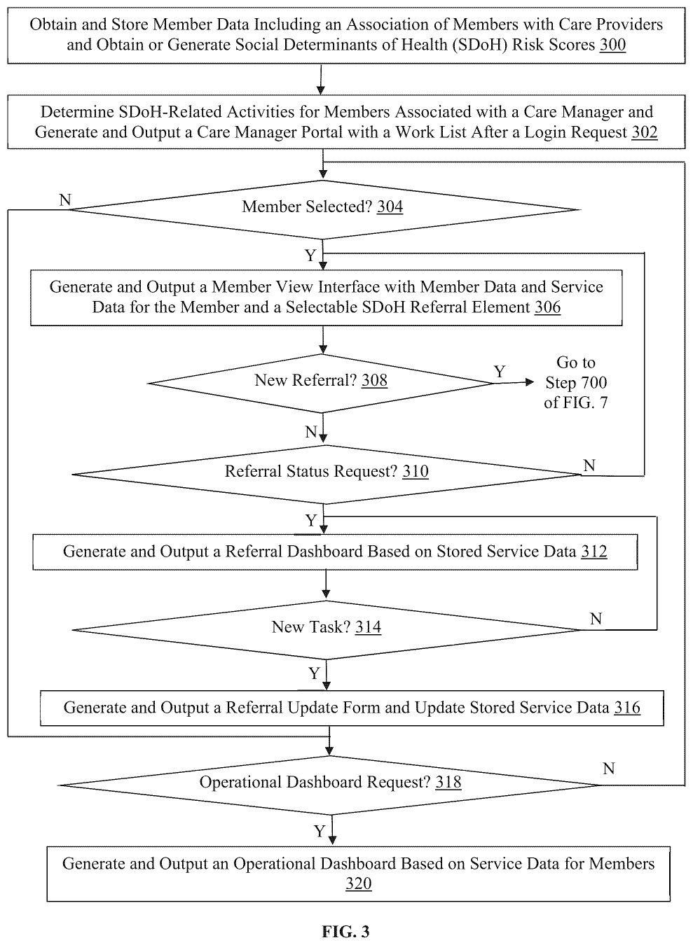

Referring now to FIG. 3, a flowchart of an exemplary method for coordinating care via the integrated SDoH platform 208 is illustrated. In step 300 in this example, the care coordination system 102 obtains and stores member data 212, which includes an association of members with care managers. In some examples, the member data is ingested from the care manager devices 110(1)-110(a) (e.g., using forms provided by the care coordination system 102 or via provided electronic files that are automatically parsed and processed) and, in other examples, the member data 212 can be ingested via an integration with an electronic medical record (EMR) system associated with a care manager, for example. Other methods of obtaining the member data can also be used.

The association of members with care managers can be based on unique identifiers for the members and the care managers. The unique identifiers for the members can be generated by the care coordination system 102 upon ingest and maintained in the member data 212. The care coordination system 102 can register the care managers based on login credentials, which are stored in the memory 202 and can include unique identifiers (e.g., usernames) for the care managers, or the unique identifiers for the care managers also can be generated by the care coordination system 102 following registration. Additionally, the association of members to care managers can be automatic based on the ingest process or manually established by care manager users of the care manager devices 110(1)-110(a), for example. The member data 212 can further include demographic data for members (e.g., patients), clinical data (e.g., lab, pharmacy, authorization, diagnosis, and/or procedure information), hospitalization and discharge data, member history data, SDoH need data, and/or any other information.

In step 300, the care coordination system 102 also obtains or generates SDoH risk scores for each of the members, which are stored in the member data 212. The SDoH risk scores can include scores for categories such as transportation, food, pharmacy, or behavioral health, and/or an overall SDoH risk score, for example. The care coordination system 102 can ingest the SDoH risk scores from a third-party source or generate the SDoH risk scores from other member data 212 associated with the particular members, for example. Accordingly, the SDoH risk scores can be indicative of a need for a particular member with respect to a particular SDoH category (e.g., transportation, food, pharmacy, or behavioral health).

Referring to FIG. 4, a screenshot of an exemplary member detail interface 400 is illustrated. The member detail interface 400 can be provided to one of the care manager devices 110(1)-110(a) for a care manager associated in the member data 212 to the member corresponding to the member detail interface 400. As illustrated in FIG. 4, the member detail interface 400 includes a health status measure, an SDoH profile with risk scores, demographic data, and insurance details, although other information can also be provided in other examples.

The health status measure in this example provides a health status score (i.e., 8 out of 10) and an indication of the top three conditions for the patient (i.e., congestive heart failure, essential hypertension, and hypercholesterolemia). The SDoH profile includes an overall SDoH risk score (i.e., 9 out of 9) along with SDoH risk scores for access to care, access to medication, housing stability, and food insecurity. Selection of one of the tabs 402 can provide additional portions of the member data 212 associated with particular member.

Referring back to FIG. 3, in step 302, the care coordination system 102 determines SDoH-related activity for members associated with a care manager and generates and outputs a care manager portal with a work list after a login request. The login request could have been received from one of the care manager devices 110(1)-110(a) as submitted by a care manager user. Upon login, the care coordination system 102 can parse the member data 212 to identify the members associated with the care manager based on a stored association of unique identifiers.

The care coordination system 102 can then determine SDoH-related activities that may be required for the members, optionally in an order of priority. The care coordination system 102 then generates a work list by analyzing demographic, clinical, and SDoH base data and/or SDoH risk scores in combination to determine next-best actions or SDoH-related activities for particular members. The care coordination system 102 then provides a care manager portal with a work list, which is optionally organized or prioritized based on the determined next-best actions or SDoH-related activities, to the one of the care manager devices 110(1)-110(a) in response to the authenticated login request. The worklist can include selectable member indications (e.g., links or buttons) for each of the members, which can be used to expand the member view and initiate an SDoH referral, as explained in more detail below.

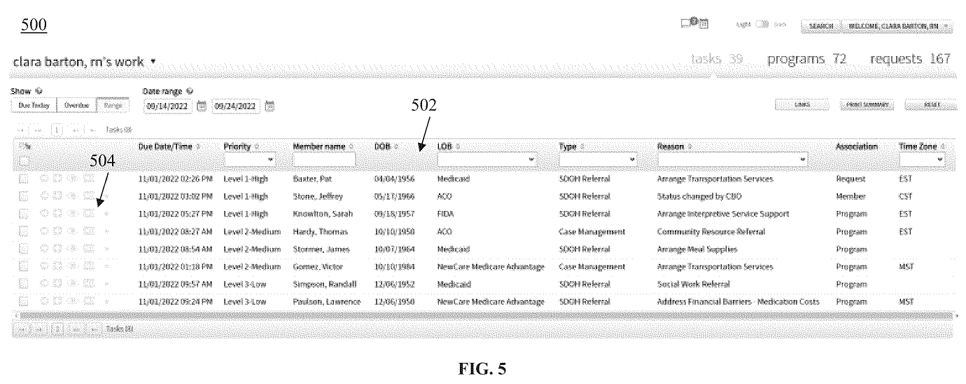

Referring to FIG. 5, a screenshot of an exemplary care manager portal 500 is illustrated. In this example, the work list 502 includes selectable member indications 504 of a plurality of members with an indication of a type of SDoH need, a reason, and an associated priority. In some examples, the member data 212, or a portion thereof, is periodically polled or retrieved from third party data sources and can indicate events such as an imminent patient discharge from a hospital, for example.

Accordingly, based on the time associated with the discharge and the current time of the login, the work list 502 may indicate a high priority for a member with respect to an SDoH referral for transportation. Other ways of determining a priority for various members can also be used in other examples. Thus, the work list 502 helps the care manager triage SDoH needs of associated members and elucidates and highlights those needs optionally according to a determined priority level and next-best action for particular members.

Referring back to FIG. 3, in step 304, the care coordination system 102 determines whether a one of the member indications for one of the members has been selected by a care manager user of one of the care manager devices 110(1)-110(a). Selection of one of the member indications for one of the members can initiate a message to the care coordination system 102. Alternatively, the care manager portal can be provided as part of the SDoH platform 208, which is configured to switch views based on received user inputs, for example. If the care coordination system 102 determines one of the member indications for one of the members has been selected, then the Yes branch is taken to step 306.

In step 306, the care coordination system 102 generates and output a member view interface with member data 212 and service data 216 for the member associated with the selected member indication, along with a selectable SDoH referral element (e.g., button or link). The member view interface can include the same and additional/different portions of the member data 212 as provided via the care manager portal, along with service data 216 indicating statuses of historical SDoH referrals (e.g., open or closed) and/or information regarding the programs associated with the historical SDoH referrals, for example.

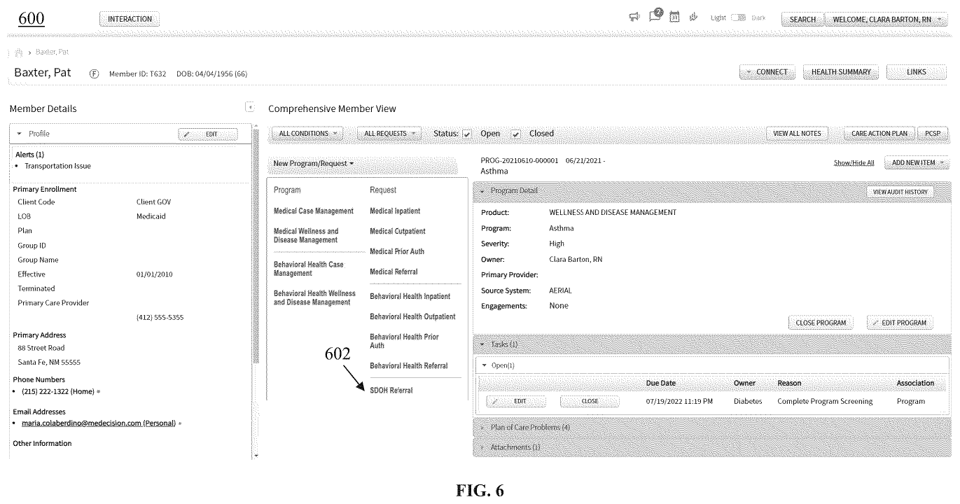

Referring to FIG. 6, a screenshot of an exemplary member view interface 600 is illustrated. In this example, the member view interface 600 includes an SDoH referral element 602, which can be selected by a care manager to initiate an SDoH referral for the particular member. Accordingly, the SDoH need (e.g., transportation, food, pharmacy, or behavioral health) can be identified through an external SDoH risk score, a generated SDoH risk score, a standardized SDoH assessment, or analytics, for example, and via selection of the SDoH referral element 602, a care manager can advantageously initiate an SDoH referral.

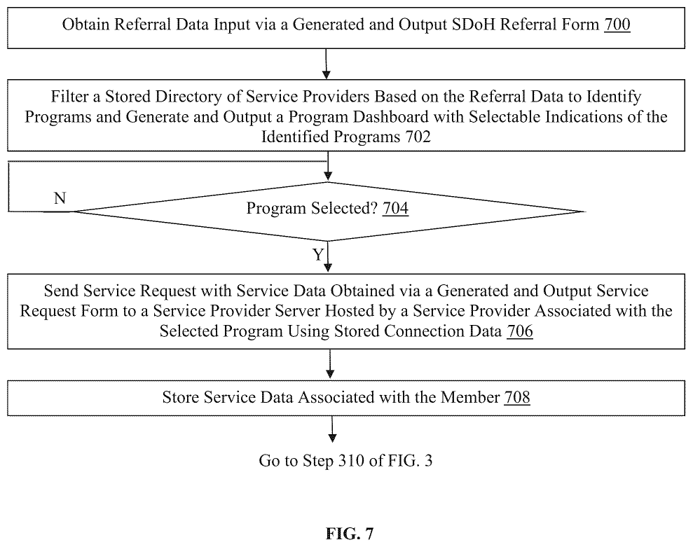

Referring back to FIG. 3, in step 308, the care coordination system 102 determines whether a new referral is requested, such as based on a selection of the SDoH referral element of the member view interface, for example. If the care coordination system 102 determines a new referral is requested, then the Yes branch is taken to step 700 of FIG. 7.

Referring now to FIG. 7, a flowchart of an exemplary method for obtaining and processing an SDoH referral to engage a service provider is illustrated. In step 700 in this example, the care coordination system 102 obtains referral data input via a generated and output SDoH referral form. Accordingly, following interaction with the SDoH referral element of the member view interface, for example, the care coordination system 102 can provide an SDoH referral form to the one of the care manager devices 110(1)-110(a) to facilitate submission of referral data. The submission of the referral data initiates a search for SDoH programs that are correlated with the referral data.

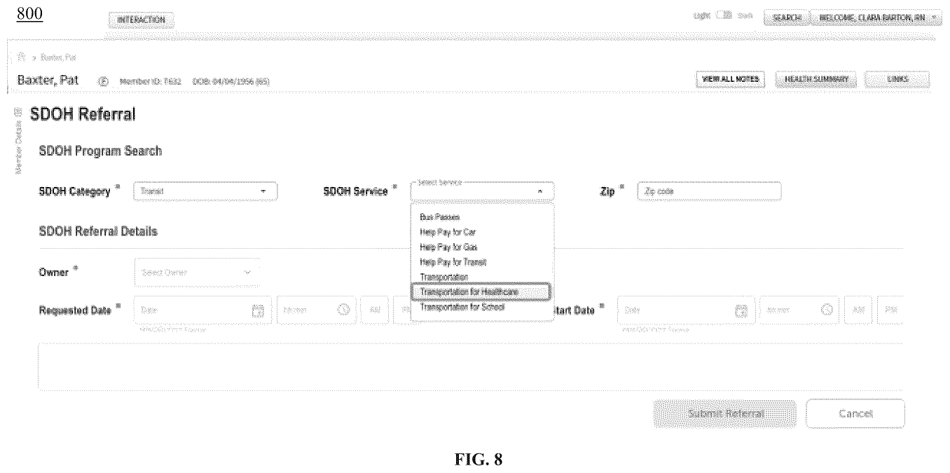

Referring to FIG. 8, a screenshot of an exemplary SDoH referral form 800 is illustrated. The SDoH referral form 800 in this example includes input fields for various types of referral data, such as a category (e.g., travel, food, pharmacy, or behavioral health), a service type (e.g., transportation to school or help pay for gas), and details regarding the SDoH service such as the location, date, and/or time, for example. Thus, in some examples, the SDoH referral form 800 collects referral data about the requested SDoH service and the information is subsequently shared with a service provider (e.g., a community-based organization (CBO)), as explained in more detail below.

Referring back to FIG. 7, in step 702, the care coordination system 102 filters a stored directory of service providers based on the referral data obtained in step 700 to identify programs and generates and outputs a program dashboard with selectable program indications of the identified programs. The directory of service providers can be maintained in the service provider data 214 and can include information regarding programs offered by various service providers, such as ride sharing programs, or pharmacy delivery programs, for example.

Accordingly, the referral data is correlated with the directory of service providers to identify responsive programs that satisfy requirements within the referral data. Optionally, the stored directory of service providers is periodically updated such that it is accurate and curated in real-time to include information from local and national CBOs, contracted network CBOs, and other service providers, for example.

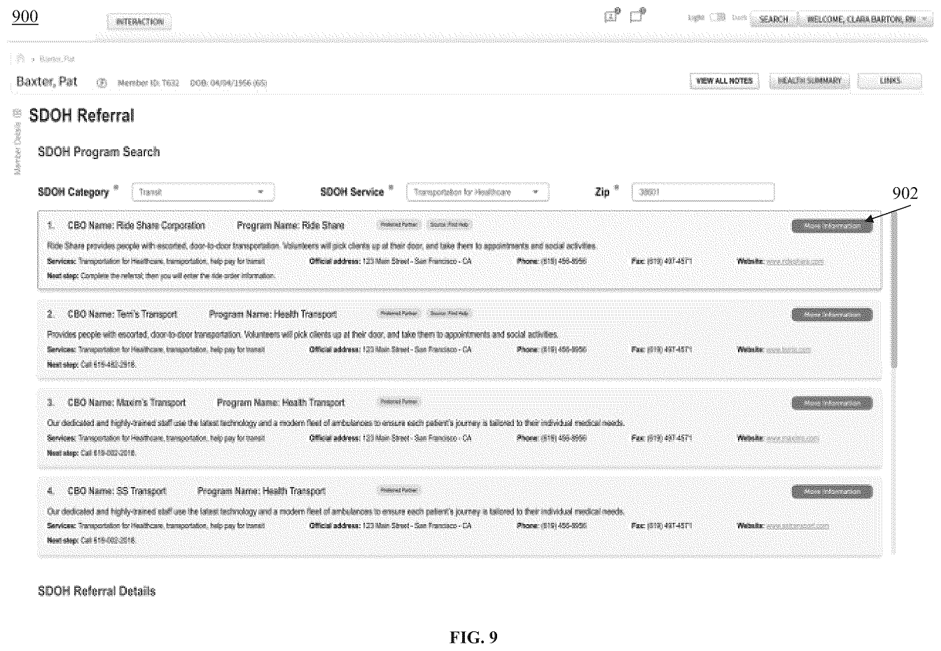

Referring to FIG. 9, a screenshot of an exemplary program dashboard 900 is illustrated. The program dashboard 900 in this example includes a listing of identified programs with a portion of the associated data extracted from the service provider data 214. For example, if the obtained referral data indicates a transit category, a transportation for healthcare service, and a particular zip code, the care coordination system 102 may identify, and display via the program dashboard 900 an indication of, ride share and health transport programs associated with ride share and transport service providers from the directory of service providers. In some examples, each program identified on the program dashboard 900 includes an associate more information button (e.g., more information button 902, selection of which can yield move information for a care manager user of one of the care manager devices 110(1)-110(a).

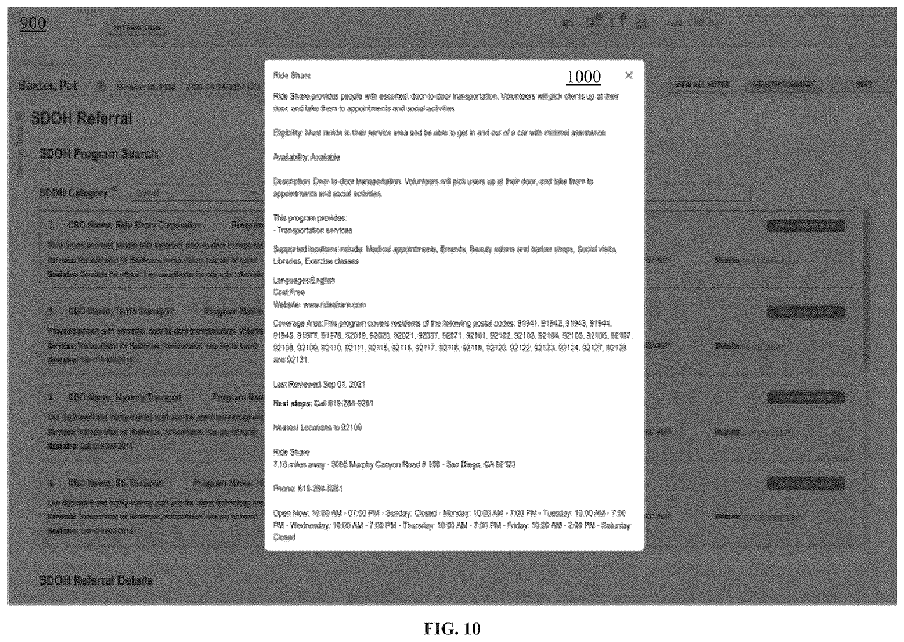

For example, referring to FIG. 10, a screenshot of the exemplary program dashboard 900 of FIG. 9 with a program detail overlay 1000 is illustrated. In this example, a care manager user of one of the care manager devices 110(1)-110(a) can select the more information button 902 to view more details about the program and the providing organization (e.g., availability of the program, coverage of the program and/or contract information associated with the program), which are retrieved from the service provider data 214 and output via the program detail overlay 1000. The additional details about the program and the CBO or other service provider help the care manager determine which program will best address the SDoH need for the particular member.

Referring back to FIG. 7, in step 704, the care coordination system 102 determines whether one of the programs is selected by the care manager user of the one of the care manager devices 110(1)-110(a). The program can be selected based on interaction by a user input device with one of the program indications on the program dashboard, for example. If the care coordination system 102 determines that a program has not been selected, then the No branch is taken back to step 704, and the care coordination system effectively waits for a program to be selected. However, if the care coordination system 102 determines a program has been selected, then the Yes branch is taken to step 706.

In step 706, the care coordination system 102 sends a service request with service data obtained via a generated and output service request form to one of the service provider servers 114(1)-114(c) hosted by a service provider associated with the selected program. In this example, the care coordination system 102 can query the service provider data 214 based on the selected program to identify connection data within the service provider data 214 for one of the service provider servers 114(1)-114(c) hosted by the associated service provider. The connection data can include API or other endpoint information, that defines a protocol or method by which the care coordination system 102 can automatically communicate with (e.g., send service data 216 to) the one of the service provider servers 114(1)-114(c).

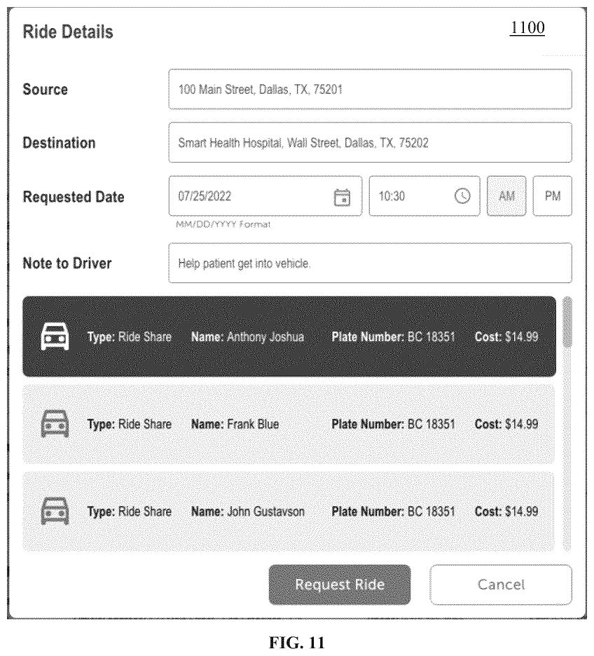

Referring to FIG. 11, a screenshot of an exemplary service request form 1100 is illustrated. In this example, service data 216 can be input via the service request form, which includes a source, destination, date, and notes for the driver for a transportation program or service. Other types of service data 216 can be obtained for different categories or types of SDoH services. Accordingly, a direct connection to a third-party service provider platform (e.g., a transportation gig economy platform) is facilitated in this example, which provides the care manager with the ability to request a service on behalf of a member immediately to close the loop on an SDoH referral and address the SDoH need in real-time.

Referring back to FIG. 7, in step 708, the care coordination system 102 stores the service data 216 associated with the member (e.g., in association with a unique identifier for the member). By storing the service data 216, the service data 216 is available for subsequent retrieval by a care manager interested in the details or status of the SDoH service or an updating by a service provider to thereby provide real-time information regarding the SDoH service, as explained in more detail below. Optionally, the member view interface 600 can be re-generated and output following submission of the service request form 1100. Subsequent to storing the service data, the care coordination system 102 proceeds to step 310 of FIG. 3 in this particular example.

Referring back to FIG. 3, in step 310, the care coordination system 102 determines whether a referral status request has been received. The referral status request can be submitted by a care manager user of one of the care manager devices 110(1)-110(a) via an interaction with the member detail interface, care manager portal, or member view interface, for example. If the care coordination system 102 determines that a referral status request has not been received, then the No branch is taken back to step 306 and the care coordination system 102 effectively waits for a subsequent interaction with the member view interface or another portion of the SDoH platform 208. However, if the care coordination system 102 determines that a referral status request has been received, then the Yes branch is taken from step 310 to step 312.

In step 312, the care coordination system 102 generates and outputs a referral dashboard based on stored service data 216. The service data 216 could have been stored as described and illustrated in more detail above with reference to step 708 of FIG. 7, for example. Accordingly, the referral dashboard can include a status of the SDoH service and SDoH program details, among other information. Optionally, the referral dashboard facilitates bidirectional communication between the care manager and a representative of the service provider.

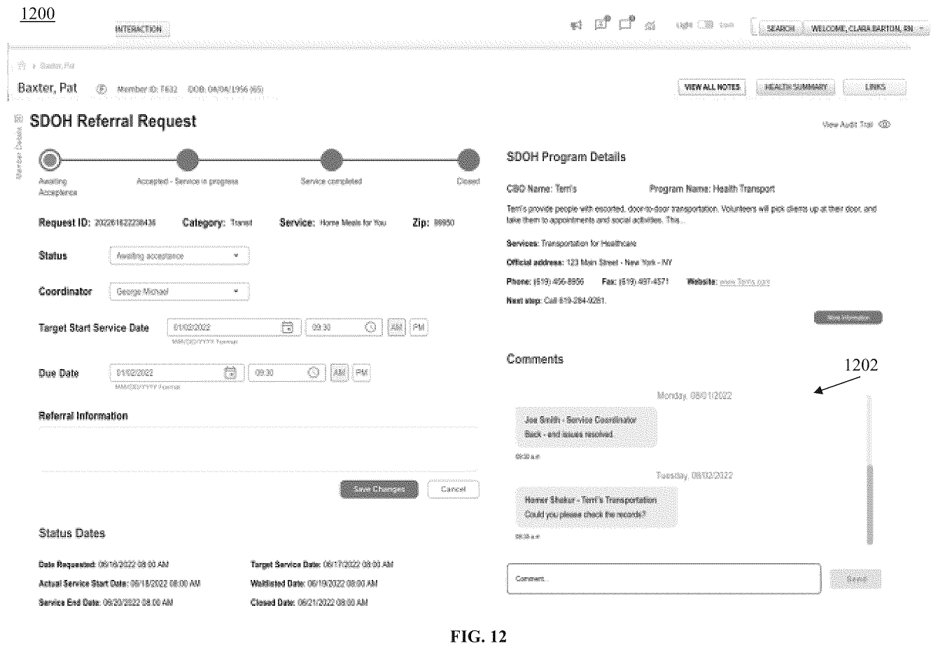

Referring to FIG. 12, a screenshot of an exemplary referral dashboard 1200 is illustrated. In this example, the SDoH referral request status is identified (e.g., awaiting acceptance, accepted-service in progress, service complete, closed). In some examples, a service provider portal can be generated and output to the service provider devices 112(1)-112(b) that facilitate input of status updates to the care coordination system 102, which updates the corresponding service data 216 in response to the status update. Additionally, the referral dashboard 1200 in this particular example includes a chat panel 1202 to facilitate electronic exchange of information, attachments, and comments between the SDoH service provider and the care manager.

Accordingly, once the SDoH Referral is submitted, it is part of the member record in the service data 216 based on an association with a unique identifier of the member, for example. The referral can then be managed together with the participating CBO or other service provider to deliver the service and close the loop all within the SDoH platform 208.

Referring back to FIG. 3, in step 314, the care coordination system 102 determines whether a new task request has been received. A new task request can be submitted by a care manager user of one of the care manager devices 110(1)-110(a) via an interaction with the care manager portal, member view interface, or referral dashboard, for example. If the care coordination system 102 determines that a new task request has not been received, then the No branch is taken back to step 312 and the care coordination system 102 effectively waits for a subsequent interaction with the referral dashboard or another portion of the SDoH platform 208. However, if the care coordination system 102 determines that a new task request has been received, then the Yes branch is taken from step 314 to step 316.

In step 316, the care coordination system 102 generates and outputs a referral update form to the one of the care manager devices 110(1)-110(a) and updates stored service data 216 based on referral update data obtained following submission of the referral update form. The referral update data can include task details, outcomes, or notes for a task associated with the program or service corresponding to an SDoH referral, although other referral update information can also be obtained in other examples. Subsequent to submission of the referral update form, the care coordination system 102 can optionally re-generate and output the referral dashboard to the one of the care manager devices 110(1)-110(a).

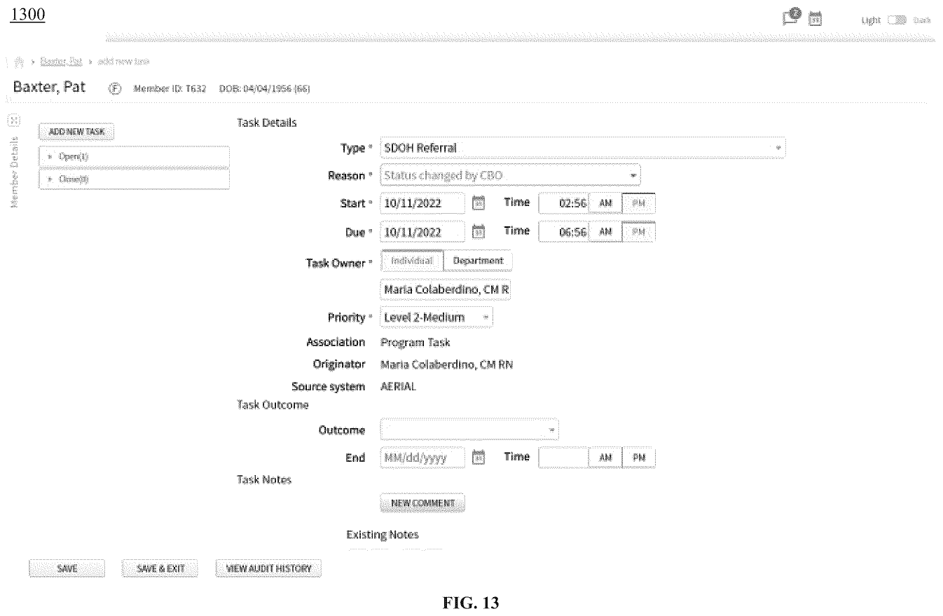

Referring to FIG. 13, a screenshot of an exemplary referral update form 1300 is illustrated. The referral update data obtained via the referral update form 1300 can replace or supplement the service data 216 associated with a prior SDoH referral. Thus, the referral update form 1300 allows tasks and notes to be added to an SDoH referral to keep other members of the care team for a member up-to-date and involved in managing the SDoH Referral.

Referring back to FIG. 3, subsequent to generating and outputting the referral update form, or if the care coordination system 102 determines in step 304 that a member has not been selected and the No branch is taken, then the care coordination system 102 proceeds to step 318. In step 318, the care coordination system 102 determines whether an operational dashboard request has been received from one of the care manager devices 110(1)-110(a). The operational dashboard request can be received via a user interaction with the care manager portal or another one of the GUIs 210 provided to the one of the care manager devices 110(1)-110(a), for example. If the care coordination system 102 determines that an operational dashboard request has not been received, then No branch is taken back to step 304 in this particular example. However, if the care coordination system 102 determines that an operational dashboard request has been received, then No branch is taken to step 320.

In step 320, the care coordination system 102 generates and outputs an operational dashboard based on service data 216 for a plurality of members associated with a particular care manager that has successfully logged into the SDoH platform 208 in the current session. In other examples, the operational dashboard can be provided to an owner or other manager of a facility with which a plurality of care managers is associated. In this example, the operational dashboard is generated by the care coordination system 102 using service data 216 for all members associated with all of the associated care managers. The operational dashboard can include graphical indications of referral volume by owner or care manager and/or by category and/or service type, for example. Accordingly, the operational dashboard includes statistical information generated from the service data 216 for a subset of care managers and/or members.

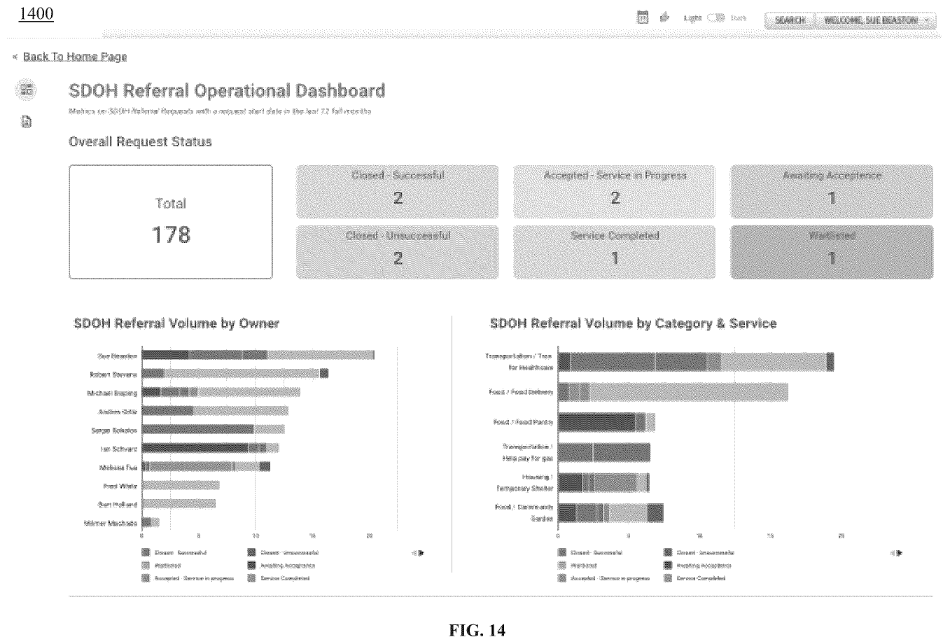

Referring to FIG. 14, a screenshot of an operational dashboard interface 1400 is illustrated. In this example, the operational dashboard interface 1400 includes real-time information regarding SDoH referrals for a plurality of members, including referrals with an associated open status, facilitates tracking of care manager effectiveness in closing the SDoH care gap, and/or highlights the CBOs or other service providers that are most involved in managing SDoH referrals, along with the most-used SDoH services, and other information can also be provided via the operational dashboard interface 1400 in other examples.

In other examples, one or more of steps 300-320 can be performed in parallel and/or in a different order for any number of users. Additionally, while in the examples explained herein the care coordination system 102 exchanges requests, responses, and other network messages with the care manager devices 110(1)-110(a) and service provider devices 112(1)-112(b), in other examples, the care coordination system 102 can provide a web application or interactive GUI (e.g., SDoH platform 208) that is configured to process a subset of those communications and generate and output for display new or modified versions of the various GUIs 210 described and illustrated herein.

Accordingly, the SDoH platform 208 and GUIs 210 of this technology more effectively and efficiently integrate social referrals into care management technology workflows to close the SDoH gap. The disclosed technology allows care managers to leverage real-time, analytics-driven insights for members based on the ingested and updated data to optimize the delivery of personalized social referrals. This technology also leverages third party integrations to provide a market of markets for clinical integration, care coordination, and patient population management to provide faster, more efficient referrals to healthcare services. Moreover, the disclosed technology uses real-time documentation of patient care and secure, bidirectional communication with automatic referral management and authorization tools to enhance care management, maximize efficiency, and close gaps in care caused by SDoH.

Having thus described the basic concept of the invention, it will be rather apparent to those skilled in the art that the foregoing detailed disclosure is intended to be presented by way of example only and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for coordinating care via an integrated social determinants of health (SDoH) platform, the method implemented by a care coordination system and comprising:
analyzing obtained demographic, clinical, and SDoH base data and generated SDoH risk scores for a plurality of members associated with a care manager to determine a next-best SDoH activity for each of the members;
generating and outputting to a care manager device a member view interface in response to a selection associated with one of the members received via a care manager interface output to the care manager device and comprising the determined next-best SDoH activity for each of the members;
obtaining referral data submitted via an SDoH referral form generated and outputted to the care manager device in response to a received selection of an SDoH referral element of the member view interface;
generating and outputting to the care manager device a program dashboard with selectable program indications for one or more programs identified by filtering a stored directory of service provider data based on the referral data; and
sending a service request to a service provider server using stored connection data in the service provider data for a service provider associated with one of the programs, in response to a received selection of one of the program indications corresponding to the one of the programs, to initiate one of the next-best SDoH activities on behalf of the one of the members.

2. The method of claim 1, further comprising generating and outputting to the care manager device a care manager portal comprising a work list comprising selectable member indications of one or more members associated with the care manager user and at least one determined SDoH-related activity for each of the members, wherein the member view interface is generated and output in response to a received selection of one of the member indications associated with the member.

3. The method of claim 1, further comprising sending the service request to the service provider server via one or more communication networks and using an application programming interface (API) endpoint.

4. The method of claim 1, further comprising:
receiving an update message from a service provider device associated with the service provider, wherein the update message comprises a status of the service initiated on behalf of the member; and
updating the stored service data based on the update message.

5. The method of claim 1, further comprising generating and outputting to the care manager device a referral dashboard based on the updated stored service data in response to a referral status request received from the care manager device.

6. A care coordination system, comprising a memory comprising programmed instructions stored thereon and one or more processors coupled to the memory and configured to execute the stored programmed instructions to:
generate and output to a care manager device via one or more communication networks for display on a display device a member view interface comprising member data for a member associated with a care manager user of the care manager device, service data associated with historical SDoH-related activities for the member, and a selectable SDoH referral element;
obtain referral data submitted via an SDoH referral form generated and output to the care manager device via the communication networks for display on the display device in response to a received selection of the SDoH referral element;
generate and output to the care manager device via the communication networks for display on the display device a program dashboard with selectable program indications for one or more programs identified by analyzing a stored directory of service provider data based on the referral data; and
send a service request via the communication networks to a service provider server associated with one of the programs using an application programming interface (API), in response to a received selection of one of the program indications associated with the one of the programs, to initiate a service on behalf of the member.

7. The care coordination system of claim 6, wherein the processors are further configured to execute the stored programmed instructions to generate and output to the care manager device via the communication networks for display on the display device a care manager portal comprising a work list comprising selectable member indications of one or more members associated with the care manager user and at least one determined SDoH-related activity for each of the members, wherein the member view interface is generated and output in response to a received selection of one of the member indications associated with the member.

8. The care coordination system of claim 6, wherein the processors are further configured to execute the stored programmed instructions to:
receive an update message from a service provider device associated with the service provider, wherein the update message comprises a status of the service initiated on behalf of the member; and
update the stored service data based on the update message.

9. The care coordination system of claim 6, wherein the processors are further configured to execute the stored programmed instructions to generate and output to the care manager device for display on the display device a referral dashboard based on the updated stored service data in response to a referral status request received from the care manager device.

10. A non-transitory computer readable medium having stored thereon instructions for coordinating care via an integrated social determinants of health (SDoH) platform comprising executable code which when executed by one or more processors, causes the processors to:
generate and output to a care manager device a member view interface comprising member data for a member associated with a care manager user of the care manager device, service data associated with historical SDoH-related activities for the member, and a selectable SDoH referral element;
obtain referral data submitted via an SDoH referral form generated and output to the care manager device in response to a received selection of the SDoH referral element, wherein the referral data comprises one or more of a service type, a service category, or a service time;
generate and output to the care manager device a program dashboard with selectable program indications for one or more programs identified by analyzing a stored directory of service provider data based on the referral data; and
send a service request to a service provider server using stored connection data in the service provider data for a service provider associated with one of the programs, in response to a received selection of one of the program indications, to initiate a service on behalf of the member.

11. The non-transitory computer readable medium of claim 10, wherein the executable code, when executed by the processors, further causes the processors to output to the care manager device for display on the display device a care manager portal comprising a work list comprising selectable member indications of one or more members associated with the care manager user and at least one determined SDoH-related activity for each of the members, wherein the member view interface is generated and outputted in response to a received selection of one of the member indications associated with the member.

12. The non-transitory computer readable medium of claim 10, wherein the executable code, when executed by the processors, further causes the processors to send the service request to the service provider server via one or more communication networks and using an application programming interface (API) endpoint.

13. The non-transitory computer readable medium of claim 10, wherein the executable code, when executed by the processors, further causes the processors to:
receive an update message from a service provider device associated with the service provider, wherein the update message comprises a status of the service initiated on behalf of the member; and
update the stored service data based on the update message.

14. The non-transitory computer readable medium of claim 10, wherein the executable code, when executed by the processors, further causes the processors to generate and output to the care manager device for display on the display device a referral dashboard based on the updated stored service data in response to a referral status request received from the care manager device.
